(19) United States
(12) Patent Application Publication
(10) Pub. No.: US 2007/0135870 A1
(43) Pub. Date: Jun. 14, 2007

(54) Method for treating hearing loss

(75) Inventors: Steven C. Shanks, Mesa, AZ (US); Kevin B. Tucek, McKinney, TX (US); Mark L. Moore, Granite Bay, CA (US); Timothy A. Chaffin, Orangevale, CA (US)

Correspondence Address: ETHERTON LAW GROUP, LLC, 5555 E. VAN BUREN STREET, SUITE 100, PHOENIX, AZ, 85008, US

(73) Assignee: HearingMed Laser Technologies, LLC

(21) Appl. No.: 11/698,667

(22) Filed: Jan. 25, 2007

Related U.S. Application Data
(63) Continuation-in-part of application No. 10/772,973, filed on Feb. 4, 2004.

(51) Int. Cl. A61N 5/06

(52) U.S. Cl. 607/89

(57) ABSTRACT

A method for treating a patient with laser energy to improve hearing loss. The method involves applying laser energy to the patient's spine, preferably by sweeping a linear laser beam over the patient's skin. The method may alternatively include applying laser energy to the patient's jaw, skull, ears, or a combination thereof. The laser device used for treating the patient is preferably a hand-held probe that moves freely relative to the patient's skin and can generate more than one wavelength of laser energy. In the preferred treatment, the patient is treated with a hand-held probe that emits two laser beams, one laser beam producing a pulsed line of red laser light and the other producing a pulsed line of green laser light. In the preferred embodiment, the patient's upper back, cervical vertebrae, cranial nerves, and temporomandibular joints are treated with laser energy for a total of less than 20 minutes in a single treatment.

### CROSS-REFERENCE TO RELATED APPLICATIONS

This application is a continuation-in-part of co-pending U.S. application Ser. No. 10/772,973 filed Feb. 4, 2004, which claims the benefit of U.S. application Ser. No. 09/932,907 filed Aug. 20, 2001, now U.S. Pat. No. 6,746,473, which claims the benefit of U.S. Provisional Application No. 60/273,282 filed Mar. 2, 2001.

### FIELD OF INVENTION

This invention relates generally to the use of laser devices for treating hearing loss. More particularly, this invention relates to a method of using a laser light device that provides warm-colored and cool-colored radiation for the treatment of hearing loss.

### BACKGROUND

Sound is collected by the pinna (the visible external part of the ear) and directed toward the eardrum through the outer ear canal. The sound makes the eardrum vibrate, which in turn causes a series of three tiny bones (the hammer, the anvil, and the stirrup) in the middle ear to vibrate. The vibration is transferred to the snail-shaped cochlea in the inner ear. The cochlea is lined with sensitive hairs which trigger the generation of nerve signals that are sent to the brain where they are interpreted as sounds (noises, speech, etc.). Sensorineural hearing loss occurs when the hair cells of the inner ear and the neural pathways to the auditory cortex are damaged. Sensorineural hearing loss accounts for about 90% of all hearing loss and is found in 23% of individuals older than 65 years of age.

Possible causes of sensorineural hearing loss include aging; acoustic trauma (injury caused by sudden loud noise); viral infections of the inner ear or auditory nerve; Méniére's disease; ototoxic drugs, such as aminoglycosides (most common cause; e.g., tobrahmycin), loop diuretics (e.g., Furosemide), antimetabolites (e.g., Methotrexate), salicylates (e.g., aspirin); acoustic neuroma; inflammation as a result of, for example, meningitis, mumps, measles, virus, syphilis, or suppurative labyrinthitis; prolonged noise exposure to loud noises; multiple sclerosis; brain tumor; stroke; physical trauma such as fracture of the temporal bone affecting the cochlea and middle ear; or congenital defect.

With the exception of surgically removing an acoustic neuroma, sensorineural hearing loss is usually irreversible. In these cases, treatment options rely on methods that amplify external sounds and on teaching the patient various strategies that essentially retrain the brain to interpret external stimuli. Primary treatment options include use of a hearing aid for one or both ears or a surgical implantation of a cochlear implant. It would be desirable to have a treatment option that is non-invasive, painless, effective, and easy to administer.

Low energy laser therapy (LLLT) is used in the treatment of a broad range of conditions. LLLT improves wound healing, reduces edema, and relieves pain of various etiologies, including successful application post-operatively to liposuction to reduce inflammation and pain. It is also used in the treatment and repair of injured muscles and tendons. LLLT was first applied to the ear as treatment of inner ear diseases by Uwe Witt, MD of, Hamburg, Germany in the 1980's. Lutz Wilden, MD, of the Center for Low Level Laser Therapy in Bad Fussing, Germany developed it further and brought it to a wide range of patients. Dr. Wilden's central thesis is that since low level laser energy is capable of penetrating targeted tissue, it is capable of stimulating the mitochondria in the corresponding underlying cells to produce energy through the production of ATP (adenosine triphosphate). Mitochondria are the power supplies of all cells; they metabolize fuel and produce energy for the cell in the form of ATP. Further, hearing impaired patients typically suffer from inflammation or atrophy of the tissues and neural pathways connected to and supporting the cochlea's cilia hair structure. Laser energy has been shown to repair damaged tissue and reduce edema. Therefore, it follows that if LLLT stimulates mitochondria to produce more energy, and reduces edema and improves wound healing, that damage to the cochlea could be repaired, thereby restoring some degree of hearing

LLLT utilizes low level laser energy, that is, the treatment has a dose rate that causes no immediate detectable temperature rise of the treated tissue and no macroscopically visible changes in tissue structure. Consequently, the treated and surrounding tissue is not heated and is not damaged. There are a number of variables in laser therapy including the wavelength of the laser beam, the area impinged by the laser beam, laser energy, pulse frequency, treatment duration and tissue characteristics. The success of each therapy depends on the relationship and combination of these variables. For example, liposuction may be facilitated with one regimen utilizing a given wavelength and treatment duration, whereas pain may be treated with a regimen utilizing a different wavelength and treatment duration, and inflammation a third regimen. Specific devices are known in the art for several types of therapy.

Recent research has shown that laser light in the cool color range excites the sympathetic subsystem of the autonomic nervous system and that laser light in the warm color range excites the parasympathetic subsystem. Other studies have shown that an imbalance in the sympathetic and parasympathetic systems impairs maximum muscle strength and nerve facilitation. It would be particularly desirable to provide both sympathetic and parasympathetic treatments with a single device to patients with hearing loss.

Therefore, an object of this invention is to provide a laser therapy method that treats hearing loss. Another object is to provide a non-invasive therapy that reduces hearing loss after a single treatment. Another object is to provide a laser therapy method that increases a patient's baseline word recognition scores after the first laser treatment. Another object to provide a laser therapy method that stimulates the cilia and cells in the cochlea to effect a physiological change that results in improved reception and interpretation of speech sounds.

### SUMMARY OF THE INVENTION

This invention is a method for treating a patient with laser energy to improve hearing loss. The method involves applying laser energy to the patient's spine, preferably by sweeping a linear laser beam over the patient's skin. The method may alternatively include applying laser energy to the patient's jaw, skull, ears, or a combination thereof. The laser device used for treating the patient is preferably a hand-held probe that moves freely relative to the patient's skin and can generate more than one wavelength of laser energy. In the preferred treatment, the patient is treated with a hand-held probe that emits two laser beams, one laser beam producing a pulsed line of red laser light and the other producing a pulsed line of green laser light. In the preferred embodiment, the patient's upper back, cervical vertebrae, cranial nerves, and temporomandibular joints are treated with laser energy for a total of less than 10 minutes in a single treatment.

### BRIEF DESCRIPTION OF THE DRAWINGS

FIG. 1 is a perspective view of the human spine, identifying the vertebrae.

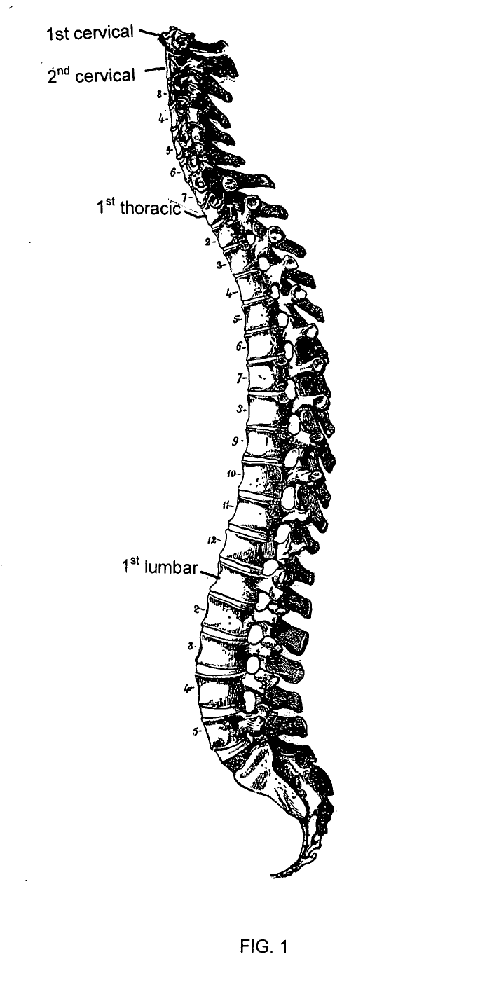

FIG. 2 is a top view of the human brain, identifying the cranial nerves.

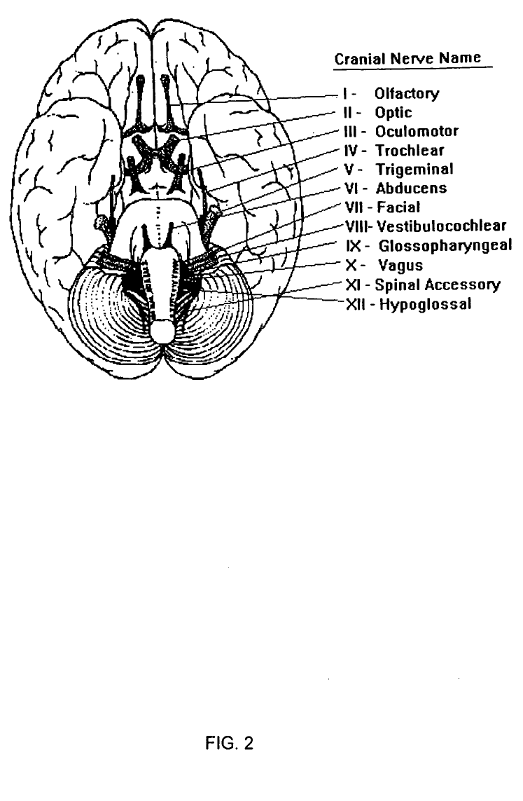

FIG. 3 is an electrical schematic illustration of a preferred embodiment of the present invention.

FIG. 4 illustrates the of application of low-level laser radiation using the preferred embodiment of the present invention.

FIG. 5 is a perspective view of a portable, floor-supported version of the preferred embodiment of the present invention.

### DETAILED DESCRIPTION OF THE INVENTION

The present invention is a method of treating hearing loss in a human patient. A principal component of this treatment is to apply laser energy to the patient's spine, preferably a selection of vertebrae including certain cervical and dorsal (thoracic) vertebrae. Optionally, laser energy may be applied to the patient's cranial nerves and temporomandibular joints. Laser energy may also be applied to one or both of the patient's ears.

In the preferred embodiment, the laser energy is applied to the patient's cervical and dorsal vertebrae C5 through T1 for about 30 seconds; to the patient's cervical vertebrae C1 through C4 for about thirty seconds; to the patient's cranial nerves C-I through C-XII for about 30 seconds; to each of the patient's temporomandibular joints for a total of about three minutes; and to each of the patient's ears, for about two minutes per ear. In the preferred embodiment, the laser energy portions are applied in the series set forth in the previous sentence, and the total application is referred to herein as the treatment. However, the order of the portions may be different in alternative embodiments. For example, the laser energy may first be applied to the patient's temporomandibular. joints, followed by laser energy application to the patient's cranial nerves and then one or more of his vertebrae. Or, the laser energy may first be applied to the patient's cranial nerves, followed by laser energy application to the patient's temporomandibular joints and then to his vertebrae C1-C4. Furthermore, one or more of the application portions may be omitted or repeated. For example, laser energy may be applied to vertebrae C5-7, but not T1. Or, laser energy may be applied to cranial nerve VIII, the vestibulocochlear nerve, in combination with laser energy application to certain vertebrae. A treatment under the present invention comprises laser energy application to one or more of the patient's vertebrae alone or in combination with one or more of the patient's cranial nerves, temporomandibular joints, or the ears. These examples herein are not an exhaustive list of the permutations of such treatment portions, but are intended to be illustrative only.

The application time for each portion, as well as the total application time, may vary from patient to patient. However, the preferred embodiment uses a total application time of less than 10 minutes. And, in the preferred embodiment, the patient is treated with only a single treatment. Consequently, a patient's word recognition can be improved with the application of certain laser energy to certain parts of the body for less than 10 minutes.

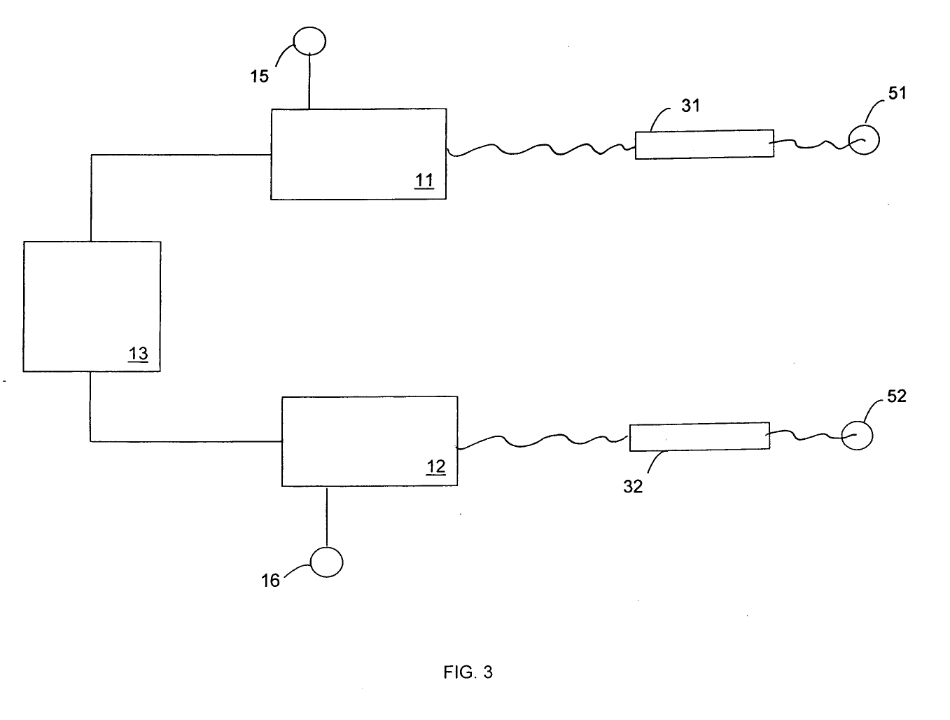

FIG. 3 shows the preferred embodiment in which a first laser energy source 11 and a second energy source 12 are connected to a power source 13. The power source preferably provides direct current, such as that provided by a battery, but may instead provide alternating current such as that provided by conventional building current that is then converted to direct current. Control circuitry is operatively connected to the laser energy sources to act as an on/off switch and control the period of time the laser light is generated. It may also be used to control pulse frequency and power. When there are no pulses, a continuous beam of laser light is generated. Pulse frequencies from 0 to 100,000 Hz may be employed to achieve the desired effect on the patient's tissue. The control circuitry can be separate components for each laser energy source, or a single control circuitry that controls all laser energy sources. In the preferred embodiment, there is a separate control circuitry 15, 16 for each laser energy source 11, 12 respectively. See FIG. 3. The laser energy sources can be energized independently or simultaneously which, throughout this specification, refers to acts occurring at generally the same time. The goal for LLLT regimen is to deliver laser energy to the target tissue utilizing a pulse frequency short enough to sufficiently energize the targeted tissue and avoid thermal damage to adjacent tissue.

Studies have shown that laser light in the warm color range, about 575-780 nm, influences largely the parasympathetic nervous system. Laser light in the cool color range, about 360-575 nm, influences largely the sympathetic nervous system. The root of the parasympathetic nervous system is primarily in the brain, upper cervical, and sacral portion of the spinal cord. The root of the sympathetic nervous system is in the thoracic and lumbar portions of the spinal cord, from level T1 to approximately L2. Thus, laser light can be used for diagnostic and therapeutic modality between the sympathetic and parasympathetic systems when applied to the appropriate nerve root(s) in the spinal cord.

Laser energy sources are known in the art for use in low-level laser therapy. They include solid state, gas, and semiconductor diode lasers. The present invention uses wavelengths from infrared to ultraviolet. The preferred embodiment uses semiconductor diode lasers which provide a broad range of wavelengths from mid-infrared to blue. The laser energy sources in the preferred embodiment are two semiconductor laser diodes. The first laser energy source 11 produces light in the red range of the visible spectrum, about 635-700 nm, and preferably 635 nm. The second laser energy source 12 produces light in the green range of the visible spectrum, about 491-575 nm, and preferably 491 nm. Other suitable wavelengths are used for other particular applications. It is advantageous to utilize at least one laser beam in the visible/UV energy spectrum so that the operator can see the laser light as it impinges the patent's body and the area treated can be easily defined. Solid state and tunable semiconductor laser diodes may also be employed to achieve the desired wavelength.

Different therapy regimens require diodes of different wattages. The preferred laser diodes use less than one watt of power each to stimulate nerve roots in the spinal cord. Diodes of various other wattages may also be employed to achieve the desired laser energy for the given regimen.

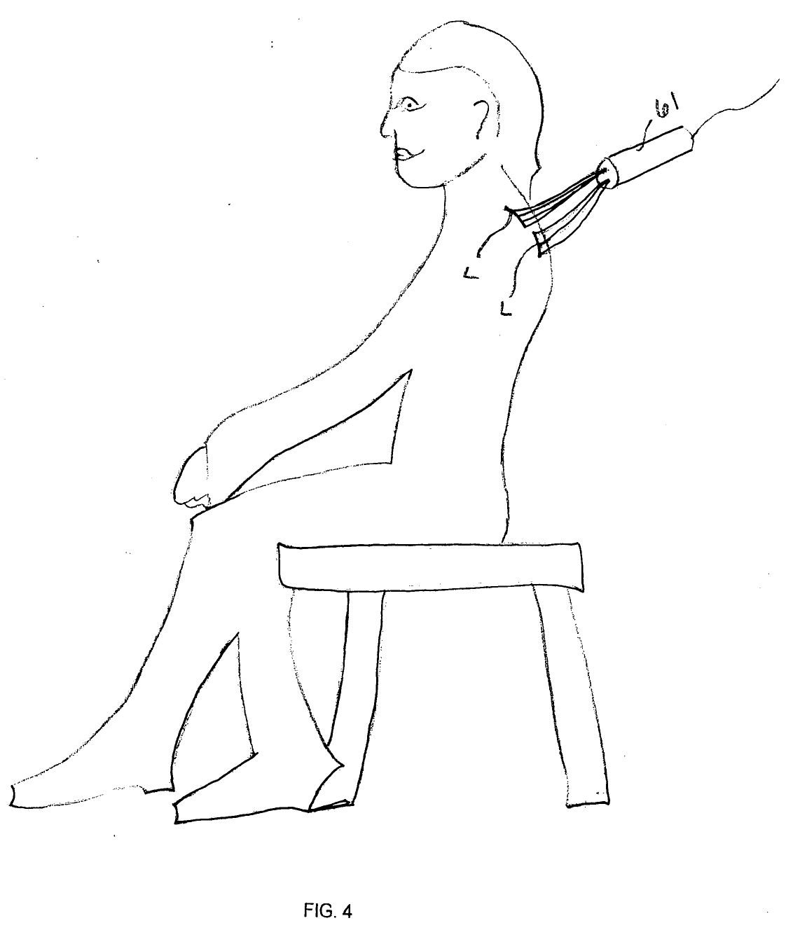

Each laser beam exits the laser and is shone through an optical arrangement to produce a beam spot. In the preferred embodiment, each laser beam 41, 42 exits the laser and is shone through an optical arrangement 31, 32, respectively, that produces a beam spots 51, 52 respectively, of a certain shape. See FIGS. 4, 5 and 6. The beam spot is the cross-sectional shape and size of the emitted beam as it exits the optical arrangement. For example, a laser beam of circular cross-section creates a circular spot as the laser light impinges the patient's skin. If the laser light emitted is in the visible range, a circular spot can be seen on the patient's skin of substantially the same diameter as the laser beam emitted from the optics arrangement. In the preferred embodiment, each laser beam passes through an optical arrangement that generates a beam of substantially linear cross-section, resulting in a line of laser light L seen on the patient's skin. See FIG. 4. In an alternative embodiment, one laser provides a linear spot L and a second laser passes through an optical arrangement that generates a beam of circular cross-section, resulting in a circular spot as seen on the patient's skin.

The optical arrangements 31 and 32 of the preferred embodiment each include a collimating lens and a line generating prism. The collimating lens and the line generating prism are disposed in serial relation to the laser energy source 11, 12 respectively. The collimating lens and the line generating prism receive and transform the generated beam of laser light into the line of laser light L. As an alternative, a rod lens can be used to generate a linear beam spot. Other optical arrangements for generating a linear beam spot include other lenses, prisms, mirrors, diffraction grating, mechanical slit, or other optical arrangement. Furthermore, a suitable electrical or mechanical arrangement could be substituted for the optical arrangement. The device may utilize as many lasers and optical arrangements as necessary to obtain the desired emissions and spot shapes. For example, the device may employ two laser diodes each with a single plain glass cover. Another example is a device employing two laser diodes each with a collimating lens and beam spot shaping lens, such that two substantially circular spot shapes are achieved. Or, for example, the device may employ two laser diodes each with an optical arrangement such that two substantially linear spot shapes are achieved. Or, in another example, more than two lasers may be used and optical arrangements aligned such that two or more of the laser beams have substantially similar spot shapes and are co-incident where they impinge the patient's skin.

The laser light can be directed to the desired area with a single hand-held wand, multiple hand-held wands, or a standalone device. FIG. 4 shows the preferred embodiment in which the laser light is emitted from a lightweight, hand-held pointer referred to herein as a wand 61. Wands are also referred to in the art as probes. The wand 61 is preferably an elongated hollow tube defining an interior cavity which is shaped to be easily retained in a user's hand. In the preferred embodiment the laser energy sources 11, 12 are mounted in the wand's interior cavity, although the laser energy sources could be remotely located and the laser light conducted by fiber optics to the wand. The wand may take on any shape that enables the laser light to be directed as needed such as tubular, T-shaped, substantially spherical, or rectangular (like a television remote control device). The housing may contain the power supply (for example a battery) or the power supply may be remote with power supplied by an electrical cable. In an alternative embodiment, the laser light is emitted from multiple wands. This enables the practitioner to apply laser light simultaneously at multiple areas on a patient's body. For example, the first wand emits green laser light and the second wand emits red laser light.

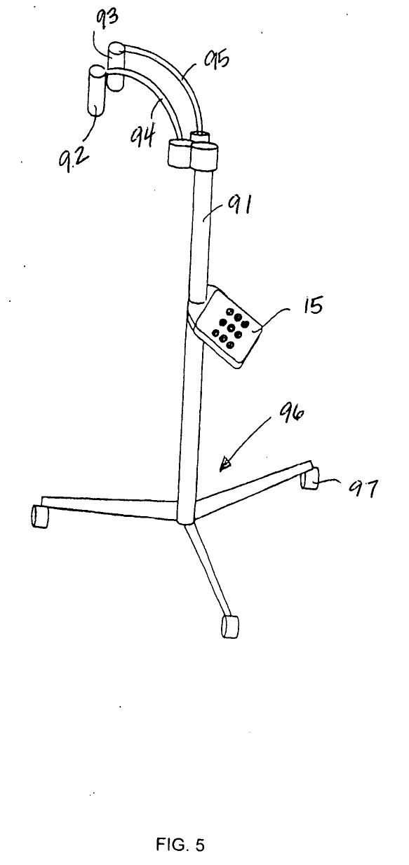

As opposed to a hand-held device, he device may operate in a stand-alone configuration. For example, the present device may be supported by a support structure such as the wall or a portable stand that rests on the floor or table. This stand-alone arrangement enables a patient to be scanned by the laser beam without movement of the housing. FIG. 5 shows the portable, floor-mounted version of the present invention. Two-housings 92 and 93 are attached to arm 91 with connectors 94 and 95, respectively. The connectors may be rigid or, preferably, flexible, so that the housings can be moved to any desired position. The arm 91 may be articulated for additional control over the position of the lasers. The arm 91 is attached to a base 96 having wheels 47 such that the device can be moved to any desired position and then stay substantially stationary while treatment is occurring. This is particularly convenient for patients lying on a table or sitting in wheelchair. Control circuitry 15 is in electrical connection with the housings and is shown in FIG. 6 mounted on the arm 41. The control, however, can be mounted elsewhere or can operate as a remote control using radio frequencies or other methods known in the art.

In yet another alternative embodiment, the laser light is emitted from an arm of a standalone device. The standalone device generally comprises the arm, a post, and a base, having sufficient weight to prevent the device from tipping. The arm is preferably an elongated hollow tube defining an interior cavity. Laser energy sources 11, 12 are mounted in the arm's interior cavity, although the laser energy sources could be remotely located and the laser light conducted by fiber optics to the arm. The arm is connected to the post, preferably in such a way that the arm is freely positionable in the x-, y-, and z-axes. Preferably house current is used as the power source in this alternative embodiment.

A shield may be employed to prevent the laser light from reflecting or deflecting to undesired locations. The shield is attached where appropriate to block the radiation. For example, the shield may be attached to the assembly, to one or more of the housings, or worn by the patient. The shield may be shaped like a canopy or helmet, but may take on other shapes, as appropriate, depending on the area to be shielded. For example, the shield may take on a rectangular or hemi-cylindrical shape to shield a patient's upper torso.

While there has been illustrated and described what is at present considered to be a preferred embodiment of the present invention, it will be understood by those skilled in the art that various changes and modifications may be made, and equivalents may be substituted for elements thereof without departing from the true scope of the invention. Therefore, it is intended that this invention not be limited to the particular embodiment disclosed as the best mode contemplated for carrying out the invention, but that the invention will include all embodiments falling within the scope of the appended claims.

I claim:

1. A method for treating hearing loss in a human patient comprising:
a) applying laser energy to at least one of the patient's vertebrae.

2. The method of claim 1 wherein the vertebra is in the range of C1 through T1.

3. The method of claim 1 wherein the laser energy emanates from a probe that moves freely relative to the skin of the patient.

4. The method of claim 1 wherein the laser energy comprises a red laser beam.

5. The method of claim 1 wherein the laser energy comprises a red laser beam and a green laser beam.

6. The method of claim 1 wherein the laser energy comprises a red laser beam and a blue laser beam.

7. A method for treating hearing loss in a human patient comprising:
a) applying laser energy to at least one of the patient's vertebrae such that the patient's word recognition improves after a single application of less than 10 minutes.

8. A method for treating hearing loss in a human patient comprising:
a) applying laser energy to at least one of the patient's temporomandibular joints.

9. The method of claim 8 wherein the laser energy emanates from a probe that moves freely relative to the patient.

10. The method of claim 8 wherein the laser energy comprises a red laser beam.

11. The method of claim 8 wherein the laser energy comprises a red laser beam and a green laser beam.

12. The method of claim 8 wherein the laser energy comprises a red laser beam and a blue laser beam.

13. A method for treating hearing loss in a human patient comprising applying laser energy to at least one of the patient's cranial nerves.

14. The method of claim 13 wherein the laser energy emanates from a probe that moves freely relative to the patient.

15. The method of claim 13 wherein the laser energy comprises a red laser beam.

16. The method of claim 13 wherein the laser energy comprises a red laser beam and a green laser beam.

17. The method of claim 13 wherein the laser energy comprises a red laser beam and a blue laser beam.

18. A method for treating hearing loss in a human patient comprising applying laser energy to the patient in the following series:
i. to the patient's vertebrae C5 through T1;
ii. to the patient's vertebrae C1 through C4; and
iii. to the patient's cranial nerves C-I through C-XII.

19. The method of claim 18 wherein the laser energy comprises a laser beam having a warm color.

20. The method of claim 19 wherein the laser beam is red.

21. The method of claim 18 wherein the laser energy comprises a laser beam having a cool color.

22. The method of claim 21 wherein the laser beam is green.

23. The method of claim 21 wherein the laser beam is blue.

24. The method of claim 18 wherein the laser energy comprises a laser beam having an ultraviolet wavelength.

25. The method of claim 18 wherein the laser energy comprises a first laser beam having a warm color and a second laser beam having a cool color.

26. The method of claim 18 wherein the laser energy comprises a laser beam producing a spot shape that is substantially linear.

27. The method of claim 18 further comprising applying laser energy to at least one of the patient's temporomandibular joints.

28. The method of claim 18 wherein:
a) the laser energy applied the patient's vertebrae C5 through T1 is applied for about 30 seconds;
b) the laser energy applied the patient's vertebrae C1 through C4 is applied for about 30 seconds;
c) the laser energy applied the patient's cranial nerves C-I through C-XII is applied for about 30 seconds; and
d) the laser energy applied to at least one of the patient's temporomandibular joint is applied for about 3 minutes.

29. The method of claim 28 further comprising applying laser energy to at least one of the patient's ears for about 2 minutes.

30. The method of claim 18 wherein the laser energy comprises a red laser beam and a green laser beam.
